(19) United States
(12) Patent Application Publication
(10) Pub. No.: US 2007/0060861 A1
(43) Pub. Date: Mar. 15, 2007

(54) Cathode for electrotransport delivery of anionic drug

(76) Inventors: Janardhanan Anand Subramony, Santa Clara, CA (US); Rama V. Padmanabhan, Los Altos, CA (US); Joseph B. Phipps, Sunnyvale, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/513,761

(22) Filed: Aug. 30, 2006

Related U.S. Application Data
(60) Provisional application No. 60/712,959, filed on Aug. 31, 2005.

(51) Int. Cl. A61N 1/30

(52) U.S. Cl. 604/20

(57) ABSTRACT

A cathode for an electrotransport drug delivery system for the delivery of anionic drugs. The cathode has a composite structure that contains an electroactive species that upon reduction does not generate any gas or an anion that competes with the anionic drug during electrotransport.

### CROSS REFERENCE TO RELATED U.S. APPLICATION DATA

The present application is derived from and claims priority to provisional application U.S. Ser. No. 60/712,959, filed Aug. 31, 2005, which is herein incorporated by reference in its entirety.

### TECHNICAL FIELD

The present invention relates to an electrotransport drug delivery system having cathode for driving anionic drugs across a body surface or membrane. In particular, the invention relates to a system having a cathode for electrotransport transdermal administration of anionic drugs across a body surface or membrane such that the cathode does not generate a competing ion for the anions being administered.

### BACKGROUND

The delivery of active agents through the skin provides many advantages, including comfort, convenience, and non-invasiveness. Gastrointestinal irritation and the variable rates of absorption and metabolism including first pass effect encountered in oral delivery are avoided. Transdermal delivery also provides a high degree of control over blood concentrations of any particular active agent.

Many active agents are not suitable for passive transdermal delivery because of their size, ionic charge characteristics, and hydrophilicity. One method for transdermal delivery of such active agents involves the use of electrical current to actively transport the active agent into the body through intact skin by electrotransport. Electrotransport techniques may include iontophoresis, electroosmosis, and electroporation. Electrotransport devices, such as iontophoretic devices are known in the art, e.g., U.S. Pat. No. 5,057,072; 5,084,008; 5,147,297; 6,039,977; 6,049,733; 6,181,963, 6,216,033, and US Patent Publication 20030191946. One electrode, called the active or donor electrode, is the electrode from which the active agent is delivered into the body. The other electrode, called the counter or return electrode, serves to close the electrical circuit through the body. In conjunction with the patient's body tissue, e.g., skin, the circuit is completed by connection of the electrodes to a source of electrical energy, and usually to circuitry capable of controlling the current passing through the device. If the ionic substance to be driven into the body is positively charged, then the positive electrode (the anode) will be the active electrode and the negative electrode (the cathode) will serve as the counter electrode. If the ionic substance to be delivered is negatively charged, then the cathodic electrode will be the active electrode and the anodic electrode will be the counter electrode.

Electrotransport devices require a reservoir or source of the active agent that is to be delivered or introduced into the body. Such reservoirs are connected to the anode or the cathode of the electrotransport device to provide a fixed or renewable source of one or more desired active agents. As electrical current flows through an electrotransport device, oxidation of a chemical species takes place at the anode while reduction of a chemical species takes place at the cathode. Typically, both of these reactions can generate a mobile ionic species with a charge state like that of the active agent in its ionic form. Such mobile ionic species are referred to as competitive species or competitive ions because the species can potentially compete with the active agent for delivery by electrotransport. For example, silver ions generated at the anode can compete with a cationic drug and chloride ions formed at the cathode can compete with an anionic drug.

Typically, the electrode material for the anode is made of silver. During electrotransport, silver is oxidized and, as a result, sliver ion is generated. At the cathode, typically AgCl (solid) is reduced to form metallic silver and chloride ion.
Ag→Ag⁺+e⁻
AgCl(s)+e⁻→Ag^{o}(s)+Cl⁻

For the delivery of cationic drugs such as fentanyl, lidocaine, metoclopramide, etc., silver (Ag) electrode in the donor compartment can act as the anode and the use of silver chloride (AgCl) as the cathode is acceptable. However, if one is interested in delivering an anionic drug, using AgCl as the cathode generates chloride ions during use. The chloride ions can compete with the anionic drug to be delivered and reduce their transport efficiency. Furthermore, silver generated at the cathode is a moderate oxidizer and can bind to proteins, which is undesirable if the anionic drug to be delivered is a peptide or a protein. Thus, silver chloride is undesirable as a cathodic material for the delivery of an anionic drug through a body surface.

The examples of Ag and AgCl as anode and cathode fall in the class of consumable electrodes, which means that the electrode material is consumed during the reaction as a function of time and has a finite lifetime. On the other hand, if a nonconsumable electrode such as a platinum or stainless steel is used as the electrode, it can generate gaseous species such as oxygen and hydrogen since it induces electrolysis of water during the reaction. Of course, any gas generation is undesirable in a reservoir or at the electrode. Some of the reactions using non-consumable electrodes are given below.

Anode
H₂O→4H⁺+O₂+4e⁻ (E⁰=1.229V)

Cathode
2H₂O+2e⁻→2OH⁻+H₂ (E⁰=−0.828V)

The generation of H⁺ and OH⁻ will also have a negative impact in that it shifts the pH of the formulation, which can in turn affect the solubility and charge state of the drug.

For the electrotransport of anionic drugs, what is needed is a cathode material that is able to undergo reduction without generating a gas or a competing ion that competes with the anionic drug.

### SUMMARY

The present invention relates to cathode materials for the electrotransport delivery of anionic drugs through a body surface (e.g., transdermally through the skin, or across an ocular tissue, such as conjunctiva or sclera). This invention identifies chemistries and methodologies to obtain cathodes for anionic delivery in iontophoretic applications without generating a gas or a competing ion. There are a number of potent drugs that are therapeutic in the anionic form for desired efficacy. The cathodic material of the present invention is applicable for delivery of many such drugs, such as cromolyn (antiasthmatic), indomethacin (anti-inflammatory), ketoprofen (anti-inflammatory) and ketorolac tromethamine (NSAID and analgesic activity). These drugs exist in the anionic form, in particular, at physiological pH. Biologics such as DNA/RNA also exist as anions.

In one aspect, the present invention provides an electrotransport system for administering an intended anion (such as a biologically beneficial drug anion) through a body surface. The system includes a cathodic reservoir containing the intended anion and a cathodic electrode for conducting a current to drive the anion. The cathodic electrode includes an electroactive substance having a metal-containing ion of a higher oxidation state. During electrotransport, the electroactive species is reduced from the higher oxidation state to a lower oxidation state without generating a gas or generating a competing ion, e.g., a non-hydroxyl anion, such as a halogen ion (e.g., chloride ion), that can compete with the anion intended or desired for electrotransport delivery.

In one aspect, the present invention provides a method making an electrotransport system for administering an intended anion (such as a biologically beneficial drug anion) through a body surface, wherein the system includes a cathodic reservoir containing the intended anion and a cathodic electrode that includes an electroactive substance having a metal-containing ion of a higher oxidation state. In another aspect, the present invention provides a method using such an electrotransport system and such an electrode.

### BRIEF DESCRIPTION OF THE FIGURES

The present invention is illustrated by way of examples in embodiments and not limitation in the figures of the accompanying drawings in which like references indicate similar elements. The figures are not shown to scale unless indicated otherwise in context.

FIG. 1 illustrates a schematic, sectional view of an embodiment of an electrode/reservoir portion of this invention.

FIG. 2 illustrates a schematic, sectional view of another embodiment of an electrode/reservoir portion of this invention.

FIG. 3 illustrates a structure of cobalamin.

FIG. 4 shows the reduction of aquocobalamin at about neutral pH.

FIG. 5A shows the reduction of hydroxocobalamin in a pH higher than neutral.

FIG. 5B shows the structure of hydroxocobalamin.

FIG. 6 illustrates how reducible cations are immobilized in ion exchange resin according to the present invention.

FIG. 7A illustrates the discharge capacity plot (voltage versus capacity) of cathode laminate consisting of 68% vitamin B12, in a polyisobutylene matrix (˜29%) with ˜3% carbon black.

FIG. 7B shows the constant iontophoretic current delivered using the vitamin B12 based cathode laminate of FIG. 7A.

FIG. 8A illustrates the discharge capacity plot (voltage versus capacity) of cathode laminate consisting of 68% hydroxocobalamin, in a polyisobutylene matrix (˜29%) with ˜3% carbon black.

FIG. 8B shows the constant iontophoretic current delivered using the hydroxocobalamin based cathode laminate of FIG. 8A.

FIG. 9 is a graph that shows the comparison of ketoprofen electrotransport on skin using hydroxocobalamin laminate electrodes versus using typical traditional AgCl laminate electrodes.

FIG. 10 is a graph that shows data of another two runs on ketoprofen electrotransport on skin using hydroxocobalamin laminate electrodes.

### DETAILED DESCRIPTION

The present invention is directed to a cathode electrode of an electrotransport drug delivery system that is able to undergo reduction without generating a gas or a competing ion that competes with an anionic drug that is intended to be delivered. In particular, the system of the present invention provides a cathodic electrode with material containing electroactive species such as transition metal ions that can undergo reduction without generating a gas or a competing ion, e.g., a halide such as chloride, that competes with the desired anion to be delivered.

The practice of the present invention will employ, unless otherwise indicated, conventional methods used by those skilled in the art in pharmaceutical product development.

In describing the present invention, the following terminology will be used in accordance with the definitions set out below.

The singular forms “a,” “an” and “the” include plural referents unless the context clearly dictates otherwise. Thus, for example, reference to “a polymer” includes a single polymer as well as a mixture of two or more different polymers.

As used herein, the term “component” refers to an element within the analgesic reservoir, including, but not limited to, an analgesic as defined above, additives, permeation enhancers, stabilizers, dyes, diluents, plasticizer, tackifying agent, pigments, carriers, inert fillers, antioxidants, excipients, gelling agents, anti-irritants, vasoconstrictors and the like.

### MODES OF CARRYING OUT THE INVENTION

The present invention provides a cathode for electrotransport delivery of anionic compounds (e.g., anionic drugs) through a surface, such as skin or mucosal membrane, e.g., buccal, rectal, behind the eye lid, on the eye such as transconjuctival or transscleral, etc.

Electrotransport devices, such as iontophoretic devices are known in the art, e.g., U.S. Pat. No. 6,216,033, can be adapted to incorporate and function with the electrodes of the present invention. The electrotransport drug delivery system typically includes portions having a reservoir associated with either an anodic electrode or a cathodic electrode (“electrode/reservoir portions”). Generally, both anodic and cathodic portions are present. The electrode/reservoir portion is for delivering an ionic drug. The electrode/reservoir portion typically includes a drug reservoir in layer form that is to be disposed proximate to or on the skin of a user for delivery of drug to the user. The drug reservoir typically includes an ionizable drug. The typical iontophoretic transdermal device can have an activation switch in the form of a push button switch and a display in the form of a light emitting diode (LED) as well. Electronic circuitry in the device provides a means for controlling current or voltage to deliver the drug via activation of the electrical delivery mechanism. The electronics are housed in a housing and an adhesive typically is present on the housing to attach the device on a body surface, e.g., skin, of a patient such that the device can be worn for many days, e.g., 1 day, 3 days, 7 days, etc. The patents disclosed above related to electrotransport are incorporated by reference in their entireties.

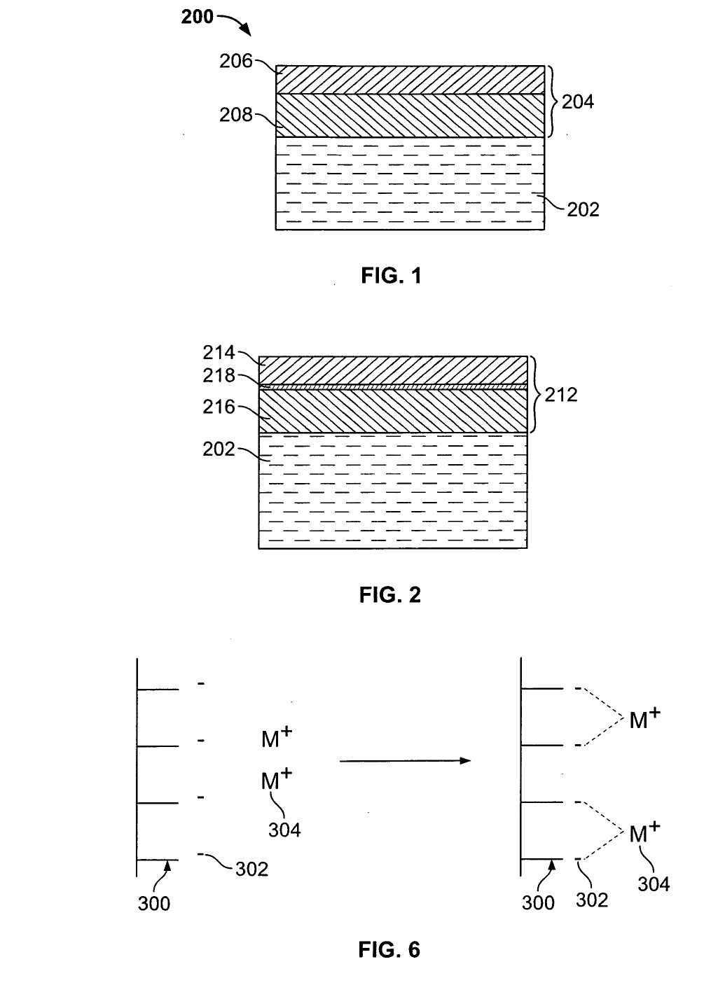

FIG. 1 shows an embodiment of an electrode/reservoir portion 200 of the present invention. The electrode/reservoir portion 200 includes reservoir 202 that contains chemical reagents (e.g., donor drug) and electrode 204 that includes a current collector 206 and oxidizable/reducible portion 208. In the embodiment in which the reservoir is an anion reservoir and the electrode 204 is a cathodic electrode, the oxidizable/reducible portion 208 is a reducible portion. In the embodiment in which the reservoir is a cation reservoir and the electrode 204 is an anodic electrode, the oxidizable/reducible portion 208 is an oxidizable portion. In summary, for cations, anode is the donor electrode and for anions, cathode is the donor electrode. Hereinafter, in describing the electrode/reservoir portions and the electrode, the case wherein an anionic agent is to be delivered from the cathode is described for illustration. It is to be understood that an embodiment of the reverse polarity can be similarly constructed except that the chemical agents are different and that the oxidation and reduction of the electrode is the opposite.

FIG. 2 shows an illustration of another embodiment in which the anode electrode/reservoir portion 210 includes reservoir 202 and electrode 212 that includes a current collector 214 and oxidizable/reducible portion 216. A conductive adhesive 218 is disposed between the metallic plate 214 and the oxidizable/reducible portion 216 and laminate them together. The cathode/reservoir portions of FIG. 1 and FIG. 2, of course, can be part of an electrotransport system with an anode, counter ion reservoir, housing that is adhesively applicable to a body surface for multiple days similar to those shown in U.S. Pat. No. 6,216,033 and the like.

To deliver an anion, the oxidizable/reducible portion 208, 216 is a reducible portion that includes chemical agents that can accept an electron and be reduced without producing either a gas (e.g., hydrogen) or a competing anion (e.g., halide ions such as chloride) that competes with the anionic agent being delivered. Any electroactive species that can undergo reduction without producing competing ions can be used. These include, transition metal ions that can exhibit multiple oxidation states such as Co, Cu, etc. Other cathodic materials include intercalation compounds such as Vanadium pentoxide, V₂O₅, metallic WO₃, tungstates, and spinels of the general structural type ABO₂ (where A and B are metals with a 2⁺ oxidation state, i.e., are divalent metals).

In one preferred aspect of the present invention, organometallic complexes having a central transition metal ion are used as the electroactive species. During electrochemical reduction, the electroactive species accepts the electron from the external circuit and undergoes reduction by changing the oxidation state and without releasing a competing ion.

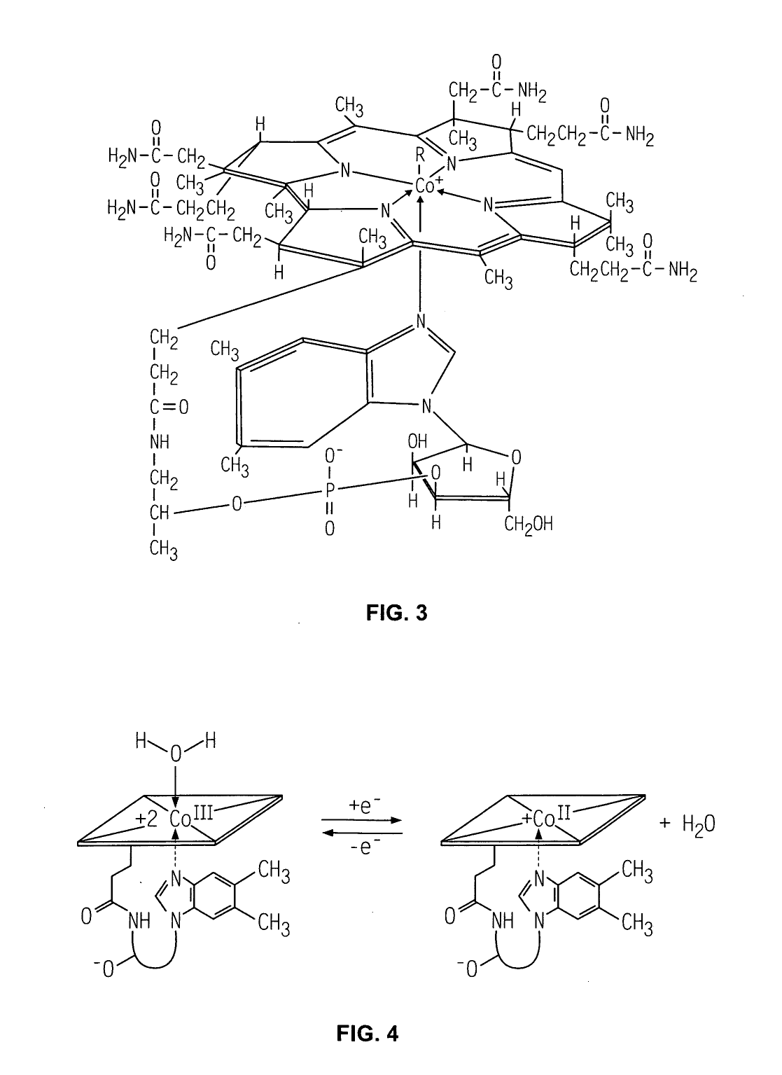

Useful complexes include organic complexes having ions of the metals such as cobalt (Co), copper (Cu), and zinc (Zn). The central metal ion could be any cation as long as it is electroactive and has multiple oxidations states that are stable. These complexes include complexes with ions of metals that have multiple (e.g., three or two) oxidation states. One such complex is cobalamin, having a structure shown in FIG. 3. In FIG. 3, R is a group or molecule such as CN, OH, H₂O, CH₃, etc. When R is CN, the cobalamin is cyanocobalamin. (vitamin B 12). The cobalt in cobalamin is at a higher oxidation state as Co(III), which upon reduction, becomes Co(II) with a lower oxidation state. Many cobalamin and cobalamin derivatives are commercially available. Another cobalt complex is B12a hydroxocobalamin (or hydroxyl cobalamin) where one of the axial ligand group is a hydroxyl group. Preferred complexes include aquocobalamin (R=H₂O), methylcobalamin (R=CH3), azidocobalamin (R=N3), adenosylcobalamin (R=5′-deoxy-5′α adenosyl), etc. Cobalt can undergo reductions from a Co(III) to Co(II) or a Co(II) to Co(I) state. FIG. 4 illustrates the reduction of aquocobalamin. A molecule of water is released in the reduction. Some of the non-cobalamin complexes of cobalt are cobalt acetyl acetonate which comes either in the III or II form, cobalt (II) phthalocyanine, Co (III) sepulchrate free base, hexamine Co (III) free base, ethylenediamine complex of cobalt, etc., which also can be used. Cobalt organic complexes have the advantage over other electroactive species in that they can undergo reduction at lower voltages. For example, with vitamin B12 (cyanocobalamin) both the Co(III)→Co(II) reaction and the Co(II)→Co (I) reaction happens at about 0.2 and 0.8V respectively versus saturated calomel electrode. See, Lexa, Doris; saveant, Jean-Michel, “The Electrochemistry of Vitamin B12”, Acc. Chem. Res. 1983, 16, 235-243. These cobalt reaction voltages are very low and similar in range to that of the Ag/AgCl reaction chemistry. It was found that vitamin B12 in electrodes showed two pairs of chemically reversible peaks in cyclic voltammetry. See, Mbindyl, et al., “Catalytic Electrochemical Synthesis Using Nanocrystalline Titanium Dioxide Cathodes in Microemulsions”, Langmuir 1998, 14, 7027-7033. The redox potential of Co (III)→to Co(II) is around −0.04V vs saturated calomel electrode. At pH of greater than 5.6 the Co(II)→Co(I) reduction takes place at −0.74V. Thus, an electrotransport device having a cathodic electrode made from cobalamin or any other organometallic cobalt (III) complex can function at low voltages of below 1V, preferably below 0.3V and does not have to operate at high voltages.

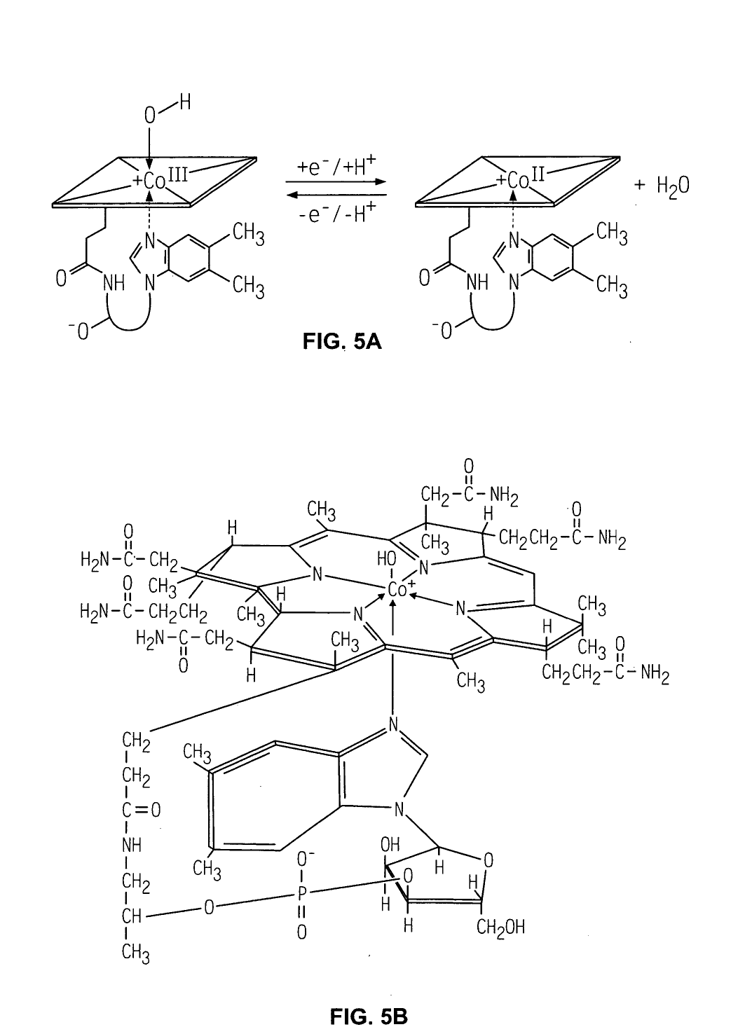

FIG. 5A shows the reduction of Co(III) in hydroxycobalamin to Co(II) with the loss of the axial water ligand. In this case, in the reduction of Co(III) in a more alkaline pH, e.g., pH8 or above, a proton is taken up by the hydroxycobalamin at a ligand site to result in the release of a water molecule from the Co complex.

FIG. 5B shows the structure of hydroxocobalamin. Thus, with the presence of cobalamin at the cathode, there is no gas or competing anion generated by the reduction process. For the delivery of anionic drugs, it is preferred that the electrode be used in a drug reservoir of pH at 7 and above, preferably pH 7-10, preferably pH 8 and above. In such an environment, the drug molecule for anionic drug delivery is in the anionic form rather than in the protonated unionized form. Generally, the ionization of a nonionic drug takes place about pH 3-5 with the ionization being substantially complete above pH 6. For example, the anionic drug ketoprofen (used for anti-inflammatory applications) remains in solution in the ionized form at basic pH and precipitates out of the formulation at acidic pH ranges. Thus, a pH of neutral and above is advantageous for using hydroxocabalamin (and similar vitamin B12 and analogs thereof) as the electroactive material for the cathode to deliver anionic drugs.

It is understood that vitamin B12 or analogs thereof can be used as long as the cobalt ions can be reduced in the electrotransport process. For example, it is contemplated that many side groups or moieties of the molecule can be substituted on the vitamin B-12 or analog molecule, thereby slightly shifting the voltages for reduction. Another approach to obtain cathodes is to incorporate metal ions at a higher oxidation state in a conductive polymeric matrix to form the cathode. Ions that are useful for this purpose include ions of metals such as Zn, Fe, Cu, Co, etc., or any transition metal cation with stable multiple oxidation states. Upon reduction, the metal ions can either attain a lower oxidation number or become reduced to zero charge, i.e., become metal, depending on their initial oxidation state.

Ferrocene, which is (bis cyclopentadienyl) iron, is another electroactive compound that can be used for the cathode electrode of the present invention. On one electron reduction the oxidation state changes from Fe (III) to Fe(II) in the cathodic reaction. Ferrocene is described by Barrette et al., “Voltammetric Evaluation of the Effective Acidities (pKa′) for Bronsted Acids in Aprotic Solvents” Analytical Chemistry (1984), 56, pp. 1890-1898.

Another class of electroactive species where no competitive ion is generated is the family of charge transfer species. In this case, the products of oxidation or reduction will generate species that are “non-competing” in that they carry a charge opposite to that of the ion to be delivered.

An example for an anodic reaction is:
[Fe(CN)₆]⁴⁻→[Fe(CN)₆]³⁻+e⁻

In the oxidation direction of the reaction, the oxidation state of Fe goes up from +2 to +3 via oxidation. Conversely, in the reduction direction the oxidation state of Fe goes from +3 to +2.

For electrotransport application, an efficient way to make metal organic complex containing cathode would be to make an electrode laminate or an electrode ink.

To make a cathode for an electrotransport device using electroactive materials, a composite electrode containing or consisting of the electroactive species, a binder and a conducting filler (to make the composite cathode electrically conductive) would be a viable approach. The composite material can either be deposited directly on a metallic plate (such as metallic plate 206 or metallic plate 214) that provides electrical connection to a current source, as shown in FIG. 1, or it can be attached to the metallic plate by a layer of adhesive. The metallic plate can, for example, be made with silver, copper, aluminum and other known electrical conductors.

Composite electrodes may be formed of a hydrophobic polymer matrix containing a conductive filler such as a powdered graphite, carbon fibers or other known electrically conductive filler material. Preferably powdered or fibrous, inert, conductive material is used. Other than carbon material, one can also incorporate electronically conducting polymers. The hydrophobic polymer based electrodes may be made by mixing the conductive filler in the hydrophobic polymer matrix. An embodiment of a process of making a reducible electrically conductive composite is as follows. The composite that is made contains a reducible material, a polymeric material and discrete conductors (such as carbon black). Powdered carbon, carbon fibers and mixtures thereof can be mixed in a hydrophobic polymer (e.g., polyisobutylene PIB) matrix, with the preferred amount of conductive filler being within the range of about 30 to 90 vol % and the remainder being the hydrophobic polymer matrix, reducible material, e.g., cobalamin or cobalt complex, in the solid form can be dispersed in heptane to prevent agglomeration and is mixed with carbon black and a polymeric material, e.g., polyisobutylene (PIB) and extruded.

In an embodiment, for example, the reducible material (cobalamin or cobalt complex) in power form is suspended in a solution of a polymer (e.g., PIB in heptane 10 wt % solids) to form a slurry and ensure good dispersion. A second slurry is made by mixing carbon black in a polymer solution, e.g., 50 vol % of carbon black on a 10 wt % PIB solution. The reducible material/polymer slurry is mixed with a carbon black/polymer/heptane slurry. The composite material can either be attached to the metallic plate while hot or be laminated by a conductive adhesive.

The slurry can also be supported on a non-woven matrix and dried, e.g., an conductive adhesive tape. Electrically conductive adhesive tapes having conductive metallic fillers are known in the art and are commercially available, for example, from Top Electronic Technology Co., Fuzhou, China or Nitto Co., Japan. If preferred, carbon black can be added directly into the reducible material/polymer slurry or the reducible material can be mixed into the carbon black/polymer slurry. A high speed mixer can be used. The amount of carbon black added is to achieve a volume resistivity of below 2000 Ω-cm, preferably 1000 Ω-cm. To achieve this resistivity, the composite generally has a ratio of polymer to carbon black of about 30 to 90 vol %, preferably 40 to 70 vol %. In an embodiment, a cationic electrode can be made with cobalamin or cobalt complex 40-70 wt %; PIB (polyisobutylene), LMMS 5-15 wt %; PIB, L-100 5-15 wt %, carbon black: 1-5 wt %. After the composite material is formed, the reducible material is present as embedded solid phase particles in the matrix of the composite material.

The polymer/carbon black/reducible material slurry can be cast on the metal plate and allowed to dry to result in a dry composite on the metal plate as an electrode. For more effective current flow, the preferred dry thick thickness of the dry composite film is about 0.1 mm to 1 mm. Alternately, the PIB/carbon black/reducible material slurry can be cast on a surface and dried to form a film, which can then be laminated to a conductive surface to form a reducible electrode.

Instead of PIB, other suitable polymers that can be used for forming the composite electrode include acrylics (or polyacrylates). It is understood that any polymer can be used in the process as long as the polymer can form a composite that allows current to flow with the desired resistivity with the presence of the included carbon black and have the structural integrity to withstand the electrotransport process. Suitable solvent that can be used is dependent on the polymer chosen. Solvents commonly used and known in the art for polymer solutions are applicable.

Another embodiment is making a conductive ink of the electroactive species such as cobalamin or cobalt complex with a binder and solvent. In this case, the electroactive species will be the cobalamin or cobalt complex and the binder can be vinyl, nitrocellulose, acrylic urethane or polyurethane based. One can add conductive materials such as carbon black in the ink for electrical conductivity of the ink.

In another embodiment of a process to make a reducible electrically conductive composite, the reducible material, e.g., cobalamin or cobalt complex, can be dissolved or dispersed in an aqueous gelling solution to form a slurry with carbon black to form the composite material with a hydrogel. The use of hydrogel containing the electroactive cathodic species might enhance the contact between the electrode material and the drug gel. The cathodic hydrogel can be made of any number of materials but preferably includes, and more preferably is made of a hydrophilic polymeric material, preferably one that is polar in nature so as to enhance the drug stability. Suitable polar polymers for the hydrogel matrix include a variety of synthetic and naturally occurring polymeric materials. A preferred hydrogel formulation contains a suitable hydrophilic polymer matrix and a preferred gelling polymer is polyvinyl alcohol, such as a washed and fully hydrolyzed polyvinyl alcohol (PVOH), e.g., MOWIOL 66-100 commercially available from Hoechst Aktiengesellschaft. Further, the description below for hydrogel in gel reservoirs can be adapted for forming the gel composite electrode.

Another embodiment is to have the complex, e.g., vitamin B12, loaded on to cation exchange resins. The cation exchange resin can either be loaded with the reducible cations before being affixed into the electrode, (such as by lamination). Vitamin B12 loaded on to cation exchange resins such as IRP-64 is commercially available. Another approach is making formulations of vitamin B12 loaded resins in aqueous hydrogels, inks or organic slurry and coating them on a suitable electrode. Making composite cathodes with complex such as cobalamin, e.g., vitamin B12, loaded in resins is yet another approach.

Cation exchange resins have carboxylic end groups and the H⁺ from a carboxylic end group can be exchanged with other cations. In the present case, the cation exchange resin would exchange the end group H⁺ with that of metal ion, e.g., cobalt complex ion, as outlined in the scheme below. The resulting resin loaded with the electroactive material can therefore be used in the cathode formulation. Vitamin B12 loaded resins are commercially available for use as nutritional supplement.

For example, vitamin B-12 can be incorporated into a cation exchange resin, e.g., AMBERLITE IRP64 available from Rohm and Haas Co. The loading of reducible cations on ion exchange resin is illustrated in FIG. 6. The polymeric matrix 300 has negative charges 302 that can attract the reduciable cations 304 to immobilize them onto the matrix.

As used herein relating to attaching the reducible cations to the negative charges of the ion exchange resin, the term “immobilizing” refers to the electrostatic bonding between the negative charge of the resin with the reducible cation. Although this electrostatic bonding can be broken, for example, by displacing the reducible cation with another cation by introducing a high concentration of the replacing cation, as long as such replacing cation is not introduced, the reducible cation remains substantially within the matrix of the ion exchange resin. Cation exchange resin useful in this invention can be a polymer having one or more acid moieties. Such acid moieties include, for example, polyacrylic acids, polyacrylic sulfonic acids, polyacrylic phosphoric acids and polyacrylic glycolic acids. Useful cation exchange resins that have negative charges in the matrix for attracting cations include, for example, AMBERLITE IRP64, from Rhom and Haas, BIO-REX 70 from Biorad, and Dowex 50 from Mallinckrodt Baker Co. of Pittsburg, N.J.

Another approach to form a composite electrode of the present invention is to form an electrode having a matrix (or carrier) that can immobilize metal ions by contacting the electrode with a solution containing the metal ions. This can be done by forming a polymeric material that has an affinity for the metal ions, followed by loading the polymeric material with the metal ions.

In one embodiment of the process, metal ions are attached in a polymer matrix that has anionic groups, such as carboxylate anion moiety, either in the backbone or side chain. An example is PLGA—(poly(D,L-lactide-co-glycolide) (PLGA), PLGA with its negatively charged carboxylate anion (COO⁻) would serve to coordinate metal ions of electroactive species such as Co of vitamin B12. Alternative to PLGA are polymeric materials that have negative charges. These include: sodium carboxy methyl cellulose, poly vinyl acetate phthalate, cellulose acetate phthalate, cellulose acetate trimaleate, ethyl acrylate methyl methacrylate, and poly ethylene terephthalate.

A conductive material can be added to this matrix to make the electrode conductive. One such approach would be to make a formulation containing PLGA with carbon black and coat it on to an electrode substrate. Once dried, the electrode can be immersed in a solution of vitamin B12 for exchange.

Cations of metals could be incorporated into these negatively charged channels/pores by immersing these electrodes in metallic salt solution overnight. The capture of metal ions in the pores would be driven by electrostatics where the carboxylates chemically attached to the polymer would coordinate with the metallic cations such as Zn²⁺. In these composite cathodic materials, during the electrotransport process, the metallic cations will be reduced to their native metallic forms with zero oxidation state (i.e., the metal). Since the carboxylates are part of the polymeric backbone, they will remain in the polymeric chain. Thus, no competing ions or gas is generated during the reduction. Cations of, for example, zinc, cobalt, iron, and copper, can be immobilized into such electrodes by incorporation into channels/pores or by ion exchange.

Cobalt based cathodes including vitamin B12 have been reported in the literature. Some examples are listed below:
Zhou, D., Njue, C. K., and Rusling, J. F. (1999) Journal of the American Chemical Society 121, pp. 2909-2914;
Aga, H., Aramata, A., and Hisaeda, Y. (1997) Journal of Electroanalytical chemistry 437, pp. 111-118;
Mbindyo, J. K. N., and Rusling, J. F. (1998) Langmuir 14, pp. 7027-7033;
Zagal, J. H., Aguirre, M. J., and Piez, M. A. (1997) Journal of Electroanalytical chemistry 437, pp. 45-52;
Shimakoshi et. al., Hydrophobic B12. Dalton Trans. (2003), pp. 2308-2312;
Ruhe, A.; Walder, L.; Scheffold, R. (1987) Makromol Chem. Macromol. Symp. 8, pp. 225-233;
Zagal, J. H.; Paez, M.; Paez, C.; J. Electroanal. Chem, 237 (1987) 145-148; and
Ariga, K., Tanaka, K., Katagiri, K., Kikuchi, J., Shimakoshi, H., Ohshimab, E., and Hisaedab, Y. (2001) Physical Chemistry Chemical Physics 3, pp. 3442-3446. Such methods of incorporating complexes, especially cobalt complexes, such as vitamin B12, onto an electrode can be adapted for making electrodes for the present invention.

It is noted that although electrodes having Vitamin B12 has been investigated in the past; such investigations were primarily on using such electrodes for catalysis in solution, in which molecules can freely move. The catalysis may be for the breakdown or synthesis of organic compounds. See, for example, Hirohide Aga, et al., Journal of Electroanalytical Chemistry 437 (1997), 111-118; J. H. Zagal, et al., Journal of Electroanalytical Chemistry 437 (1997), 45-52; De-Ling Zhou et al., J. Am. Chem. Soc. 1999, 121, 2909-2914; and Hisashi Shimakoshi et al., The Royal Society of Chemistry Chem. Commun., 2004, 50-51, all of which are incorporated by reference herein. Often times, in such catalysis reactions, gases are involved, either as a reactant or as a product of the reaction. In the present invention, the electrode (more specifically the cation, e.g., cobalt ion in the complex compound, in the electrode) does not act as a catalyst but is there to undergo redox reaction to enable current flow and ion migration in the reservoir. No gas is involved since gases are undesirable in the reservoir, as they would tend to create channels in the reservoir of the electrotransport system.

The reservoir of the electrotransport delivery devices generally contains a gel matrix, with the drug solution uniformly dispersed in at least one of the reservoirs. Gel reservoirs are described, e.g., in U.S. Pat. Nos. 6,039,977 and 6,181,963, which are incorporated by reference herein in their entireties. Suitable polymers for the gel matrix can comprise essentially any nonionic synthetic and/or naturally occurring polymeric materials. A polar nature is preferred when the active agent is polar and/or capable of ionization, so as to enhance agent solubility. Optionally, the gel matrix can be water swellable. Examples of suitable synthetic polymers include, but are not limited to, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(vinyl alcohol) and poly(allyl alcohol). Hydroxyl functional condensation polymers (i.e., polyesters, polycarbonates, polyurethanes) are also examples of suitable polar synthetic polymers. Polar naturally occurring polymers (or derivatives thereof) suitable for use as the gel matrix are exemplified by cellulose ethers, methyl cellulose ethers, cellulose and hydroxylated cellulose, methyl cellulose and hydroxylated methyl cellulose, gums such as guar, locust, karaya, xanthan, gelatin, and derivatives thereof. Ionic polymers can also be used for the matrix provided that the available counterions are either drug ions or other ions that are oppositely charged relative to the active agent.

In certain embodiments of the invention, the reservoir of the electrotransport delivery system can contain a polyvinyl alcohol hydrogel, such as that which has been described, for example, in U.S. Pat. No. 6,039,977. Polyvinyl alcohol hydrogels can be prepared, for example, as described in U.S. Pat. No. 6,039,977. The weight percentage of the polyvinyl alcohol used to prepare gel matrices for the reservoirs of the electrotransport delivery devices, in certain embodiments of the methods of the invention, is about 10% to about 30%, preferably about 15% to about 25%, and more preferably about 19%. Preferably, for ease of processing and application, the gel matrix has a viscosity of from about 1,000 to about 200,000 poise, preferably from about 5,000 to about 50,000 poise.

Incorporation of the drug solution into the gel matrix in a reservoir can be done in any number of ways, i.e., by imbibing the solution into the reservoir matrix, by admixing the drug solution with the matrix material prior to hydrogel formation, or the like. In additional embodiments, the drug reservoir may optionally contain additional components, such as additives, permeation enhancers, stabilizers, dyes, diluents, plasticizer, tackifying agent, pigments, carriers, inert fillers, antioxidants, excipients, gelling agents, anti-irritants, vasoconstrictors and other materials as are generally known to the transdermal art. Such materials can be included by one skilled in the art.

### EXAMPLES

### Example 1

Electrode laminates of vitamin B12 were prepared by mixing vitamin B12 with polyisobutylene (PIB) and carbon black. Two molecular weight grades of PIB were used as the binder and carbon black was used for making the laminate conductive. The following table summarizes a weight percent range for the composition of the film. The materials were initially dispersed in heptane and the laminate was obtained by double coating a composition onto a Reemay 2250 non-woven polyester fabric by solvent casting.

14.58+/−0.78 w/w L100 (High MW PIB)

14.58+−0.78 w/w LMMS (Low MW PIB)

68.27+−0.78 w/w vitamin B12

2.56+/−0.08 w/w Carbon Black

The characterization of the vitamin B12 laminate was carried out through discharge capacity test.

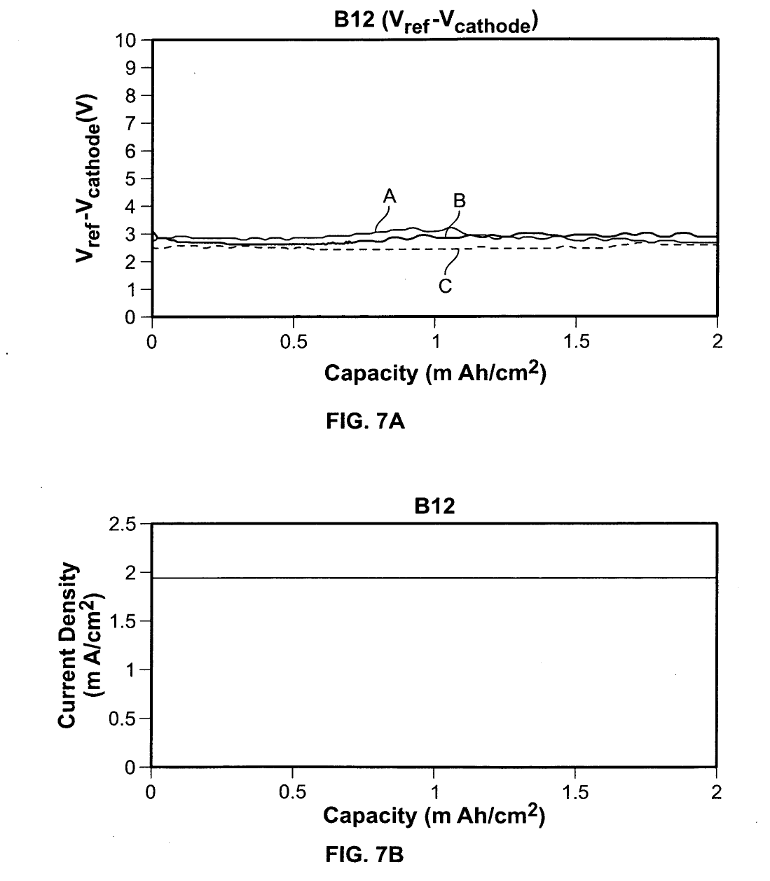

Chronopotentiometry is the technique used to determine the discharge capacities. Constant current is applied between a working and a counter electrode, and the potential of a working electrode, relative to a reference electrode, is observed. The scan usually begins at the open circuit potential, where no current flows. The potential reaches the steady state and keeps constant until the entire working electrode is consumed. Discharge capacity is defined as the duration in hour (h) where the voltage is maintained constant multiplied by the applied current (mA) per unit surface area (cm²) of the working electrode and is expressed in mAh/cm². The discharge capacity can also be expressed as mAh/g. FIG. 7A shows the discharge capacity data in which the working electrode was a vitamin B12 laminate, the reference electrode was Ag/AgCl and the counter electrode was Ag. In FIG. 7A, the voltage (in V relative to the reference) is plotted against the discharge capacity (mAh/cm²). The size of the reservoir through which the current was applied was typical of a transdermal electrotransport device. The thickness was about 11.5 mil (0.29 mm). The surface area through which the current was applied was about 1 cm². The reservoir contained a hydroxylethyl cellulose based aqueous gel (3-10% HEC containing about 0.1M NaCl).

Three different electrodes (n=3) were tested over a period of at least 12 hours of continuous activity. The data of three runs in FIG. 7A are represented by tracings A, B, and C. In a patient controlled delivery system the activation by the patient is intermittent. Thus, such an electrode can be used for many days—e.g., two days or up to a week. FIG. 7B shows the current density in mA/cm² versus the discharge capacity at the vitamin B12 electrodes of FIG. 7A. Very little fluctuation in the voltage and no fluctuation were seen while using the vitamin B12 laminate as cathodes. The current was maintained at 2 mA/cm² till a capacity of 2 mAh/cm². This is comparable to the performance using a silver chloride (AgCl) electrode. In FIG. 7B, because the currents of the three runs were stable and the same, the results of the three runs superimpose on one another in FIG. 7B. This result demonstrated that a functional electrode suitable for electrotransport (anionic drug delivery) can be made with cobalt complexes (in this Example, vitamin B12). Thus, electrotransport systems with such electrodes can be made and are expected to be functional in delivery anionic drugs.

### Example 2

Electrode laminates of vitamin B12 analog hydroxocobalamin were prepared by mixing hydroxocobalamin with polyisobutylene (PIB) and carbon black. Two molecular weight grades of PIB were used as the binder and carbon black was used for making the laminate conductive. The following table summarizes a weight percent range for the composition of the film. The materials were initially dispersed in heptane and the laminate was obtained by double coating a composition onto a Reemay 2250 non-woven polyester fabric by solvent casting.

14.58+−0.78 w/w L100 (High MW PIB)

14.58+−0.78 w/w LMMS (Low MW PIB)

68.27+/−0.78 w/w hydroxocobalamin

2.56+/−0.08 w/w Carbon Black

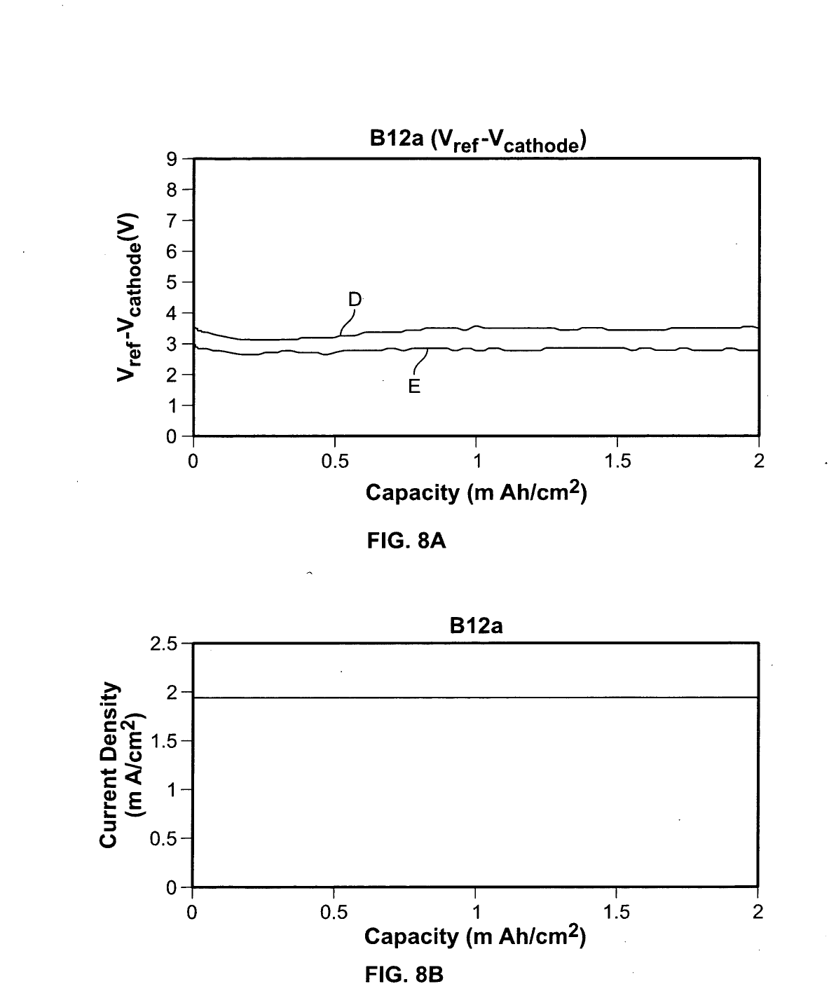

FIG. 8A and FIG. 8B show the discharge capacity data in which the working electrodes were each a vitamin B12a (hydroxocobalamin) laminate, the reference electrode was Ag/AgCl and the counter electrode was Ag. The electrode was similar to that of Example 1 except for the difference of hydroxocobalamin from cyanocobalamin. The electrodes were tested over a period of at least 12 hours of continuous activity. FIG. 8A shows the voltage (tracings D and E for two runs) and FIG. 8B shows the current density versus the discharge capacity at the hydroxocobalamin electrodes in two runs (n=2). Very little fluctuation in the voltage and no fluctuation in current density were seen while using the hydroxocobalamin laminate as cathode. The current density was maintained at 2 mA/cm² till a capacity of 2 mAh/cm². This is comparable to the performance using a AgCl electrode. This result demonstrated that a functional electrode suitable for electrotransport can be made with a cobalt complex, such as hydroxocobalamin. Thus, electrotransport systems with such electrodes can be made and are expected to be functional to deliver anionic drugs. It is further expected other cobalamin or cobalt complexes made into cathodes will produce similar results. It is further expected that other organometallic complexes will be able to be made into cathodes that will be useful in a similar manner. It is also expected that other materials having transitional metal at a reducible oxidation state will be able to be made into cathodes that will be useful in a similar manner.

### Example 3

### Electrotransport of Ketoprofen Through Cadaver Skin

Electrode laminates of vitamin B12 analog hydroxocobalamin were prepared as in Example 2. The electrode laminates were tested for flux with cadaver skin. The working electrodes were each vitamin B12a (hydroxocobalamin) laminate, and the counter electrode was Ag. The electrodes were tested over a period of time with continuous activity for flux of ketoprofen (an anionic drug), which has the following structure:

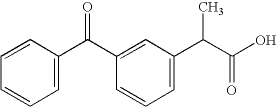

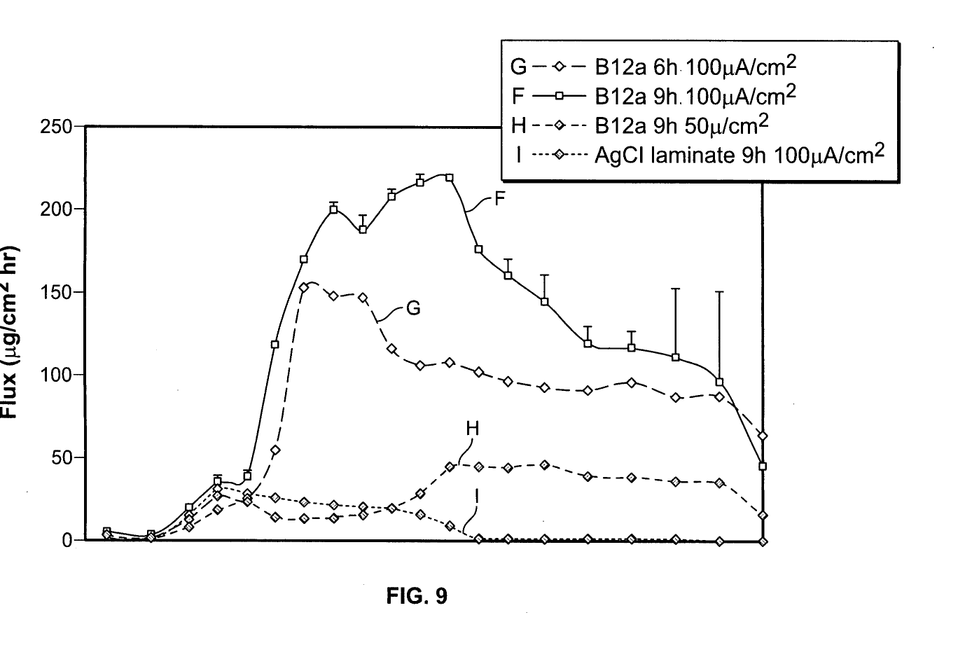

FIG. 9 is a graph that shows the comparison of ketoprofen flux on cadaver skin using hydroxocobalamin laminate electrodes versus using a typical traditional AgCl laminate electrode of similar dimensions. During a test, a constant current density was run for a period of time to determine the flux as a function of time. Curve F is the curve showing the data of a run with a hydroxocobalamin laminate electrode for 9 hours at a current density of 100 μA/cm². Four electrodes (n=4) were done for the run of Curve F. Curve G (involving 4 electrodes, n=4) is the curve of another run with a hydroxocobalamin laminate electrode for 6 hours at a current density of 100 μA/cm². Curve H (involving 2 electrodes, n=2) is the curve of a run with a hydroxocobalamin laminate electrode for 9 hours at 50 μA/cm². Curve I (involving 4 electrodes, n=4) is the curve of a run with a hydroxocobalamin laminate electrode for 9 hours at 100 μA/cm². FIG. 9 shows that the hydroxocobalamin laminate electrodes performed well compared to typical standard AgCl electrodes of similar dimensions.

The following methodology was used for the in vitro flux experiments for illustrative purposes.

Custom-built horizontal diffusion cells made in-house from DELRIN® polymeric material were used for the in vitro skin flux experiments and heat separated human epidermis was used. A cathode electrode with the same polarity as the anionic drug was adhered to one end of a DELRIN® material diffusion cell that functioned as the donor cell. The counter electrode made of Ag was adhered at the opposite end. These electrodes were connected to a current generator (Maccor) that applied a direct current across the cell. The Maccor unit was capable of applying a voltage up to 20V to maintain constant iontophoretic current.

In a typical experiment, the heat separated human epidermis was punched out into suitable circles of 24 mm ( 15/16 in) diameter and refrigerated just prior to use. The skin was placed on a screen 24 mm ( 15/16 in) that fitted into the midsection of the DELRIN® housing assembly. Underneath the screen was a small reservoir that was 13 mm (½ in) in diameter, 1.6 mm ( 1/16 in) deep and could hold approximately 250 μl of receptor solution. The stratum corneum side of the skin was placed facing the drug-containing hydrogel and the epidermis side faced the receptor reservoir. The receptor solution (saline, phosphate or other buffered solutions compatible with the drug) was continuously pumped through the reservoir via polymer tubing (Upchurch Scientific) connected to the end of a syringe/pump assembly. The drug containing polymer (gel) layer was placed between the donor electrode and heat separated epidermis. A custom-built DELRIN® spacer was used to encase the drug layer such that when the entire assembly was assembled together, the drug-containing polymer was not pressed too hard against the skin as to puncture it. Double-sided sticky tape was used to create a seal between all the DELRIN® parts and to ensure there were no leaks during the experiment. The entire assembly was placed between two heating blocks that are set at 37° C. to replicate skin temperature.

A Hanson Research MICROETTE™ collection system, interfaced to the experimental setup, collected the drug containing receptor solution from the reservoir underneath the skin directly into HPLC vials. The collection system was programmed to collect samples at specified time intervals depending on the length of the flux experiment, for example, at every hour for 24 hours. The Hanson system collected samples to be analyzed by an HPLC to determine delivery efficiency of the drug in the formulation.

A 1/10 diluted Delbeccos phosphate buffered saline (DPBS) receptor solution was used as the receiver fluid because it had the same concentration as the endogenous fluid. The DPBS was pumped into the receptor solution reservoir at 1 ml/hr. After the gel containing the drug was prepared, the drug-containing polymeric gel material was placed in the donor compartment next to the donor electrode for the test.

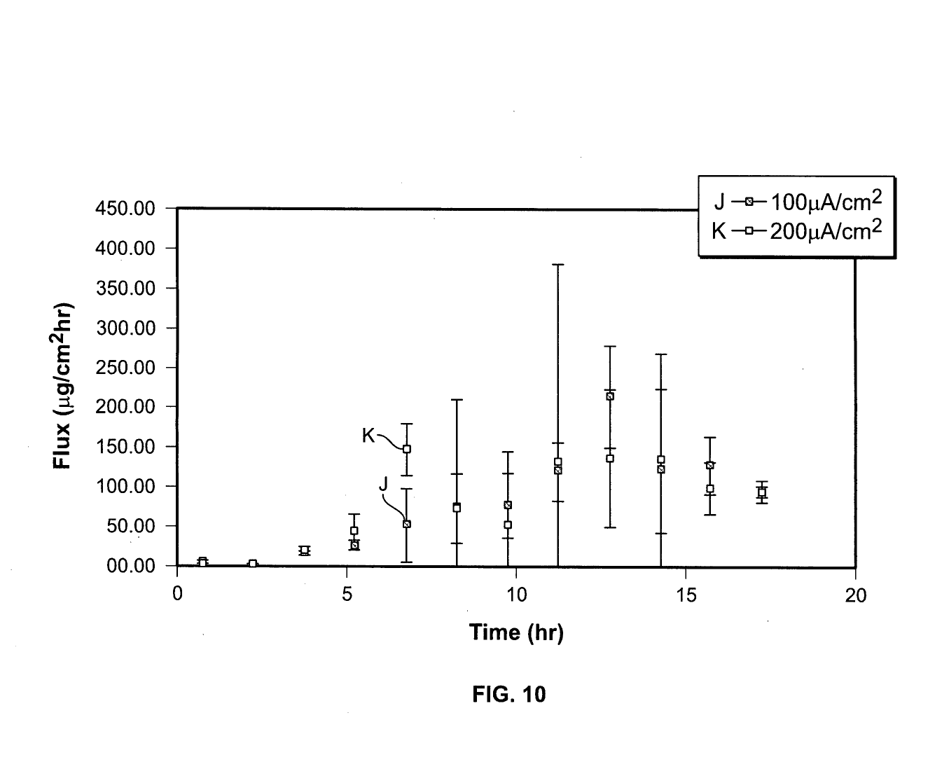

FIG. 10 is a graph that shows another two runs with hydroxocobalamin laminate electrodes in electrotransport of ketoprofen through cadaver skin. Hydroxocobalamin laminate electrodes were made and tested with procedures similar to those of FIG. 9. Run J (involving 3 electrodes, n=3) was done with a constant density of 100 μA/cm². Run K (involving 3 electrodes, n=3) was done with a constant density of 200 μA/cm². The vertical lines extending from the central data point symbols represent the standard deviation values. FIG. 10 illustrates that the hydroxocobalamin laminate electrodes can be used to achieve flux similar to what was depicted in FIG. 9. Thus, FIGS. 9 and 10 show that hydroxocobalamin laminate electrodes can be used to achieve flux through skin comparable to that from traditional AgCl electrodes.

The above-described exemplary embodiments are intended to be illustrative in all respects, rather than restrictive, of the present invention. Thus the present invention is capable of many variations in detailed implementation that can be derived from the description contained herein by a person skilled in the art. All such variations and modifications are considered to be within the scope of the present invention. The entire disclosure of each patent, patent application, and publication cited or described in this document is hereby incorporated herein by reference. Embodiments of the present invention have been described with specificity. It is to be understood that various combinations and permutations of various parts and components of the schemes disclosed herein can be implemented by one skilled in the art without departing from the scope of the present invention.

We claim:

1. A electrotransport system for administering a biologically beneficial anion for electrotransport through a body surface, comprising:
(a) cathodic reservoir comprising the biologically beneficial anion;
(b) cathodic electrode for conducting a current to drive the biologically beneficial anion in the cathodic reservoir, the cathodic electrode having a cation of a higher oxidation state, the cation during electrotransport being reducible from the higher oxidation state to a lower oxidation state without generating from the electrode a halide ion that competes with the biologically beneficial anion and without generating a gas in the cathodic reservoir.

2. The system of claim 1 wherein the cation is an ion in a complex compound or a metal ion.

3. The system of claim 2 wherein the cation is an ion of a complex compound and the electrode can provide stable current and voltage for at least 2 mAh/cm².

4. The system of claim 2 wherein the cation contains cobalt.

5. The system of claim 2 wherein the cation is cobalt (III) in the higher oxidation state.

6. The system of claim 2 wherein the cation is a metal ion and is immobilized on a porous polymeric substrate on the electrode.

7. The system of claim 2 wherein the cation is a metal ion selected from the group consisting of ions of zinc, cobalt, iron, and copper.

8. The system of claim 2 wherein the cation is immobilized in a polymer having negative charges.

9. The system of claim 2 wherein the cation is immobilized in a cation exchange material.

10. The system of claim 2 further comprising an anode electrode and an anodic reservoir.

11. The system of claim 2 further comprising an anode electrode and an anodic reservoir and has a housing that is adhesively wearable on the body surface of a patient for multiple days.

12. The system of claim 2 wherein the reducible cation is embedded as part of a solid in a polymeric matrix.

13. The system of claim 2 wherein the reducible cation coordinates either with water or with a hydroxyl group such that water is released by the reduction of the cation.

14. The system of claim 2 wherein the cation is embedded as part of a solid in a polymeric matrix and the system further having a conductive nonreducible powder embedded in a polymeric matrix.

15. The system of claim 2 wherein the cation is a cobalt complex ion of cobalamin.

16. The system of claim 2 wherein the cation is a complex ion of a complex compound and does not act as a catalyst during electrotransport.

17. A method of making an electrotransport system for administering a biologically beneficial anion for electrotransport through a body surface, comprising:
using an electroactive substance to make a cathodic electrode for conducting a current to drive the biologically beneficial anion, the electroactive substance having a cation of a higher oxidation state, the cation during electrotransport being reducible from the higher oxidation state to a lower oxidation state without generating from the electrode a halide ion that competes with the biologically beneficial anion and without generating a gas in a cathodic reservoir having the biologically beneficial anion; and
electrically connecting the cathodic electrode to the cathodic reservoir.

18. The method of claim 17 comprising using one of an ion in a complex compound and a metal ion as the cation.

19. The method of claim 18 comprising using one of an ion of a complex compound immobilized in the electrode or a metal ion immobilized in the electrode as the cation and the electrode can provide stable current and voltage for at least 2 mAh/cm².

20. The method of claim 18 wherein the cation contains cobalt.

21. The method of claim 18 wherein the cation is cobalt (III) in the higher oxidation state.

22. The method of claim 18 comprising immobilizing the cation on a porous polymeric substrate on the electrode and wherein the cation is a metal ion.

23. The method of claim 18 wherein the cation is a metal ion selected from the group consisting of ions of zinc, cobalt, and copper.

24. The method of claim 18 further comprising using a cation that coordinates either with water or with a hydroxyl group such that water is released by the reduction of the cation.

25. The method of claim 18 further comprising using cobalt complex ion of cobalamin as the cation.

26. The method of claim 18 further comprising embedding the cation as part of a soild in a polymeric matrix and embedding a conductive nonreducible powder in the polymeric matrix.

27. The method of claim 18 wherein the cation is a complex ion of a complex compound and does not act as a catalyst during electrotransport.

28. A method of electrotransport to drive a biologically beneficial anion from a reservoir into an ion permeating medium, comprising:
providing a cathodic electrode for conducting a current to drive the biologically beneficial anion, whereby the electrode has a cation of a higher oxidation state, the cation during electrotransport being reducible from the higher oxidation state to a lower oxidation state without generating from the electrode a halide ion that competes with the biologically beneficial anion and without generating a gas;
electrically connecting the cathodic electrode to a cathodic reservoir having the biologically beneficial anion, and
completing electrical circuit with the cathodic electrode, the reservoir, the medium and an anode to cause current flow into the cathodic electrode to drive the biologically beneficial anion from the cathodic reservoir into the medium in contact with the cathodic reservoir such that the cation is reduced to a lower oxidation state.
